(19) United States
(12) Patent Application Publication
(10) Pub. No.: US 2017/0240485 A1
(43) Pub. Date: Aug. 24, 2017

(54) Synthesis of Esters by Functionalisation of CO2

(71) Applicant: Commissariat a L'Energie Atomique et aux Energies Alternatives, Paris (FR)

(72) Inventors: Thibault Cantat, Issy Les Moulineaux (FR); Xavier Frogneux, Arcueil (FR)

(73) Assignee: Commissariat a L'Energie Atomique et aux Energies Alternatives, Paris (FR)

(21) Appl. No.: 15/519,791

(22) PCT Filed: Oct. 21, 2015
(86) PCT No.: PCT/IB2015/058114

§ 371 Date: Apr. 17, 2017

(30) Foreign Application Priority Data
Oct. 21, 2014 (FR) 1460132

(51) Int. Cl. C07B 41/12; C07D 333/40; C07C 67/00; C07D 213/803

(52) U.S. Cl. CPC C07B 41/12; C07D 213/803; C07D 333/40; C07C 67/00

(57) ABSTRACT

The invention relates to a method for (I) producing a carboxylic ester of formula (I). Said method comprises the steps of: a) bringing an organosilane/borane of formula Si or B into contact with CO₂, in the presence of a catalyst and an electrophilic compound of formula (III), the groups R¹, R², R³, R⁴, R⁵, Y, and M′ being as defined in claim 1; and optionally b) recovering the compound of formula (I) produced.

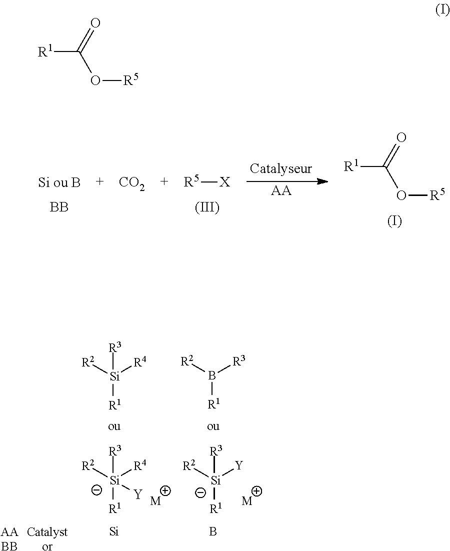

The invention relates to a process for the preparation of carboxylic esters of formula (I) and to the use of this process for the recovery in value of CO₂ emissions.

The use of CO₂ which can be recovered in value as carbon source for the production of chemical consumables is a priority challenge in order to reduce its accumulation in the atmosphere but also in order to control our dependency on fossil fuels. The greatest challenge faced by scientists and industrialists is to recycle CO₂, that is to say to develop reactions which make it possible to produce chemical compounds (fuels, plastic polymers, medicaments, detergents, high-tonnage molecules, and the like) conventionally obtained by petrochemical methods. The technical difficulty lies in the development of chemical reactions which make it possible to functionalize CO₂ by the formation of a new C—C bond (i.e., by inserting, into a C—Si bond, CO₂ by its carbon atom).

In view of the high thermodynamic stability of carbon dioxide, its conversion to give novel chemical consumables necessarily involves an external energy source so as to promote the thermodynamic balance of the chemical conversion. Today, all the efforts of the scientific community are focused on the use of electricity or light to carry out the electroreduction or the photoreduction of CO₂ to give formic acid, methanal, methanol and methane (Morris, A. J., Meyer and G. J., Fujita, E., Accounts Chem. Res., 2009, 42, 1983). A recent paper describes that the use of silanes makes it possible to reduce CO₂ under organocatalytic conditions (Riduan, S. N., Zhang, Y. G. and Ying, J. Y., Angewandte Chemie-International Edition, 2009, 48, 3322). The authors describe the formation of silylated products of formyl (SiOCHO), acetal (SiOCH₂OSi) and methoxide (SiOCH₃) type. While this strategy is justified by the importance of the reduction products of CO₂ in the chemical industry (HCOOH, H₂CO, CH₃OH), these molecules are currently used on a scale which remains very low with respect to the available amount of CO₂ which can be recovered in value. In other words, if these molecules were produced exclusively from CO₂, they would make possible the recovery in value, in view of the current market, of only 3.4% of the CO₂ produced each year which can be recovered in value (2.5 Gt/year) (Panorama des voies de valorisation du CO₂ [Overview of the routes for the recovery in value of CO₂], ADEME, June 2010). Thus, there exists a real need to diversify the number of chemical consumables which may be obtained from CO₂.

Another strategy consists in using a reactive (of high energy) chemical partner to promote the thermodynamic balance of the reaction. This strategy has been gradually spreading in recent years on the scientific scene but much progress still remains to be made in order to open up on a large scale the supply of molecules available from CO₂. The only industrial processes based on this approach are the synthesis of urea obtained by condensation of ammonia with CO₂ (equation 1) (Sakakura, T., Choi and J. C., Yasuda, H., Chem Rev., 2007, 107, 2365) and the synthesis of salicylic acid (equation 2) by the Kolbe-Schmitt process. According to the same principle, the synthesis of polycarbonates by CO₂/epoxides copolymerization is in the course of being operated industrially (equation 3) (Panorama des voies de valorisation du CO₂ [Overview of the routes for the recovery in value of CO₂], ADEME, June 2010). In this example, there is no formal reduction of the carbon center of CO₂.

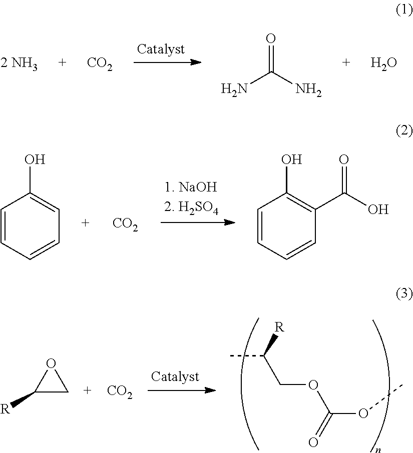

In order to produce molecules today which result from the petrochemical industry, it is necessary to overcome a technical challenge: to couple the functionalization of CO₂ with a chemical reduction stage.

A few studies relating to the synthesis of carboxylic esters from CO₂ have thus been reported in the literature. This is because esters are a class of chemical compounds which are important in the chemical industry, where they are used as solvents, reactants, food additives, for perfumery and medicinal active principles and they constitute one of the fundamental reactants of the chemistry of plastics.

The esterification of terminal alkynes by CO₂ has been developed by several groups, using catalysts based on Cu(I) or on Ag(I). The presence of a stoichiometric amount of base makes it possible to form the organocuprate, which subsequently undergoes the insertion of CO₂. The presence of an electrophilic entity will release the ester and make possible the regeneration of the catalyst. Mention may be made, among the publications based on this principle, of the following reference: Bing Yu, Zhen-Feng Diao, Chun-Xiang Guo, Chun-Lai Zhong, Liang-Nian He, Ya-Nan Zhao, Qing-Wen Song, An-Hua Liu and Jin-Quan Wang, Green Chem., 2013, 15, 2401-2407.

Furthermore, the synthesis of cyclic esters (lactones) with CO₂ has been developed using a catalyst based on Ag(I), in the presence of an excess of DBU (1,8-diazabicyclo[5.4.0]undec-7-ene), with substrates exhibiting an alkyne functional group in the γ position with respect to a carbonyl functional group (Satoshi Kikuchi, Kohei Sekine, Tomonobu Ishida and Tohru Yamada, Angew. Chem. Int. Ed., 2012, 51, 6989-6992).

However, these processes involve several stages which require intermediate purifications, which accordingly reduces the energy yield of the conversion.

There has now been developed a novel reaction which makes it possible to convert CO₂ into a carboxylating agent for the formation of carboxylic esters of formula (I):

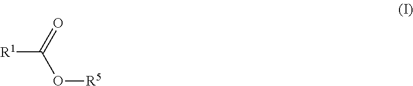

in which R^{l} and R⁵ are as defined below.

This process advantageously makes it possible to obtain, under mild conditions, in a single stage and with an excellent selectivity, a wide range of esters by recovering CO₂ in value. According to this process, the organosilane/borane makes it possible to fix the CO₂ by the formation of intermediates of carboxylate type which are trapped by an electrophilic reactant, such as an alkyl halide.

The ester compounds are thus obtained with a good yield, of the order of 35 to 100%, for example, and an excellent selectivity, generally greater than 70%.

Thus, according to a first subject matter, the invention relates to a process for the preparation of a carboxylic ester of formula (I):

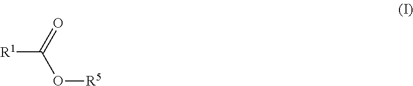

in which:

R¹ independently represents a C₁-C₁₂ alkyl group, a C₂-C₁₂ alkenyl group, a C₂-C₁₂ alkynyl group, a C₆-C₁₀ aryl group, a (C₆-C₁₀)aryl(C₁-C₄)alkyl group, a 5- to 7-membered heteroaryl group, a 5- to 7-membered heterocycle, a silyl group —Si(R⁶)₃, a siloxy group —Si(OR⁶)₃ or an amino group —NR⁷R⁸, said alkyl, alkenyl, alkynyl, aryl, arylalkyl, heteroaryl and heterocycle groups optionally being substituted by one or more R⁹ groups,

R⁵ independently represents a C₁-C₁₂ alkyl group, a C₂-C₁₂ alkenyl group, a C₂-C₁₂ alkynyl group, a C₆-C₁₀ aryl group, a (C₆-C₁₀)aryl(C₁-C₄)alkyl group, a 5- to 7-membered heteroaryl group, a 5- to 7-membered heterocycle, a silyl group —Si(R⁶)₃, a siloxy group —Si(OR⁶)₃ or an amino group —NR⁷R⁸, said alkyl, alkenyl, alkynyl, arylalkyl, aryl, heteroaryl and heterocycle groups optionally being substituted by one or more R⁹ groups,

said process comprising the stages of:

a) bringing an organosilane/borane of formula Si or B into contact with CO₂ in the presence of a catalyst and of an electrophilic compound of formula (III):

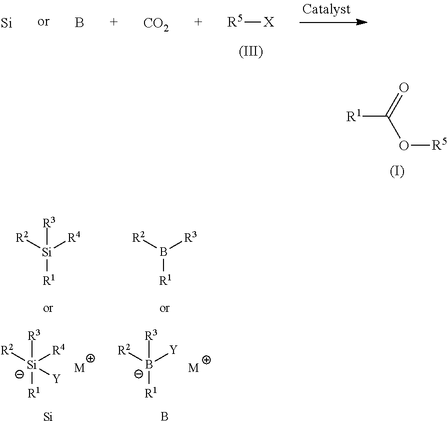

in which:

R², R³ and R⁴ represent, independently of one another, a C₁-C₁₂ alkyl group, a C₂-C₁₂ alkenyl group, a C₂-C₁₂ alkynyl group, a C₁-C₁₂ alkoxy group, a C₆-C₁₀ aryl group, a 5- to 7-membered heteroaryl group, a 5- to 7-membered heterocycle, a silyl group —Si(R⁶)₃, a siloxy group —Si(OR⁶)₃ or an amino group —NR⁷R⁸, said alkyl, alkenyl, alkynyl, aryl, heteroaryl and heterocycle groups optionally being substituted by one or more R¹⁰ groups;

Y represents a negatively charged organic or inorganic ligand, for example a halide, alkoxide, phenoxide, tosylate or mesylate, cyanide, nitrite, nitrate or carbonate;

M represents an organic or inorganic cation, in particular chosen from quaternary ammonium, alkali metal, alkaline earth metal, sulfonium or phosphonium ions;

X represents a halogen atom Cl, Br, I or F, or an —OSO₂R¹¹ group, or else R⁵—X, taken in its entirety, represents an oxonium salt, for example trimethyloxonium tetrafluoroborate or triethyloxonium tetrafluoroborate;

R⁶, in each case, is independently chosen from a hydrogen atom, a halogen atom, a C₁-C₆ alkyl group, a C alkoxy group or a C₆-C₁₀ aryl group;

R⁷ and R⁸ are each independently chosen from a hydrogen atom, a C₁-C₆ alkyl group, a C₂-C₆ alkenyl group, a C₂-C₆ alkynyl group, a C₆-C₁₀ aryl group, a 5- to 7-membered heteroaryl group, a 5- to 7-membered heterocycle, a silyl group —Si(R⁶)₃ or a siloxy group —Si(OR⁶)₃;

R⁹ and R¹⁰ are, in each case, independently chosen from a halogen atom, a C₁-C₆ alkyl group, a C₁-C₆ perfluoroalkyl group, a hydroxyl group, a C₁-C₆ alkoxy group, a nitro (—NO₂) group, a nitrile (—CN) group or a C₆-C₁₀ aryl group;

R¹¹ in each case, independently chosen from a C₁-C₆ alkyl or perfluoroalkyl group or a C₆-C₁₀ aryl group, said aryl group optionally being substituted by one or more C₁-C₆ alkyls,

and optionally

b) recovering the compound of formula (I) obtained.

According to one aspect of the invention, R¹ is a C₆-C₁₀ aryl group or a 5- to 7-membered heteroaryl group.

According to another aspect of the invention, R⁵ is a C₁-C₁₂ alkyl group, a C₂-C₁₂ alkenyl group, a C₆-C₁₀ aryl group or a (C₆-C₁₀)aryl(C₁-C₄)alkyl group.

According to yet another aspect of the invention, R², R³ and R⁴ represent, independently of one another, a C₁-C₁₂ alkyl group, a C₂-C₁₂ alkenyl group or a C₁-C₁₂ alkoxy group.

According to yet another aspect, X represents Br or I.

According to one embodiment, the catalyst is an organic catalyst, a metal salt or a metal complex.

The organic catalyst can be chosen from the following entities:
nitrogenous bases, such as, for example, secondary or tertiary amines chosen from triazabicy clodecene (TBD); N-methyltriazabicy clodecene (MeTBD), 1,8-diazabicyclo[5.4.0]undec-7-ene (DBU), trimethylamine, triethylamine, piperidine, 4-dimethylaminopyridine (DMAP), 1,4-diazabicyclo[2.2.2]octane (DABCO), proline, phenylalanine, a thiazolium salt or N,N-diisopropylethylamine (DIPEA or DIEA);
phosphorus-comprising bases, such as, for example, alkyl- and arylphosphines chosen from triphenylphosphine, 2,2′-bis(diphenylphosphino)-1,1′-binaphthyl (BINAP) or triisopropylphosphine; alkyl and aryl phosphonates chosen from diphenyl phosphate, triphenyl phosphate (TPP), tri(isopropylphenyl) phosphate (TIPP), cresyl diphenyl phosphate (CDP) or tricresyl phosphate (TCP); alkyl- and arylphosphates chosen from di(n-butyl) phosphate (DBP), tris(2-ethylhexyl) phosphate or triethyl phosphate; or Verkade bases chosen from 2,8,9-triis opropyl-2,5,8,9-tetraaza-1-phosphabicyclo[3.3.3]undecane, 2,8,9-triisobutyl-2,5,8,9-tetraaza-1-phosphabicyclo[3.3.3]undecane or 2,8,9-trimethyl-2,5,8,9-tetraaza-1-phosphabicyclo[3.3.3]undecane;
carbon-comprising bases for which the protonation takes place on a carbon atom, such as, for example, a N-heterocyclic carbene, such as a carbene resulting from an imidazolium salt, for instance 1,3-bis(2,6-diisopropylphenyl)-1H-imidazol-3-ium, 1,3-bis(2,6-diisopropylphenyl)-4,5-dihydro-1H-imidazol-3-ium, 1,3-bis(2,4,6-trimethylphenyl)-1H-imidazol-3-ium, 1,3-bis (2,4,6-trimethy 1phenyl)-4,5-dihydro-1H-imidazol-3-ium, 4,5-dichloro-1,3-bis(2,6-diisopropylphenyl)-1H-imidazol-3-ium, 1,3-di(tert-butyl)-1H-imidazol-3-ium or 1,3-di(tert-butyl)-4,5-dihydro-1H-imidazol-3-ium salts, said salts being, for example, in the form of chloride salts, as represented below:

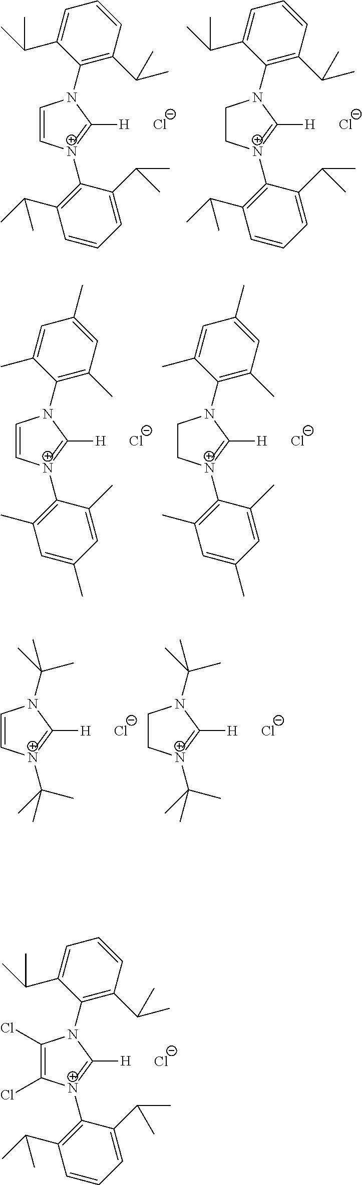

organic fluoride salts, such as TBAT (tetrabutylammonium triphenyldifluorosilicate), TASF (tris(dimethylamino)sulfonium difluorotrimethylsilicate), tetrabutylammonium fluoride or tetramethylammonium fluoride,
or
oxygen-comprising bases, such as, for example, hydrogen peroxide; benzoyl peroxide; or an alkoxide, such as the methoxide, ethoxide, propoxide, butoxide, pentoxide or hexoxide, of sodium or potassium.

The catalyst is preferably a metal salt or complex and can be chosen from the salts or complexes of:
metalloids of Groups 13-16, such as, for example, boron, silicon, aluminum, gallium, tin or indium;
alkali metals, such as, for example, sodium or potassium;
alkaline earth metals, such as, for example, magnesium or calcium;
transition metals, such as, for example, nickel, iron, cobalt, zinc, copper, rhodium, ruthenium, platinum, palladium or iridium,
rare earth metals, such as, for example, lanthanum, cerium, praseodymium, neodymium.

Preferably, the catalyst is a salt or complex of a metalloid of Groups 13-16, or of a transition metal. Preferably, the metal is silicon or copper.

The metal complex is understood to mean an organometallic or inorganic coordination compound in which a metal ion is bonded to an organic or inorganic ligand. An organometallic or inorganic complex can be obtained by mixing a metal salt with a ligand, the latter being bonded to the metal via phosphorus, sulfur, carbon, nitrogen, oxygen, hydrogen or silicon atoms, for example.

The ligands which can be brought together with the metal salt can be chosen from the following entities:
nitrogenous bases, such as, for example, secondary or tertiary amines chosen from triazabicyclodecene (TBD); N-methyltriazabicyclodecene (MeTBD), 1,8-diazabicyclo[5.4.0]undec-7-ene (DBU), trimethylamine, triethylamine, piperidine, 4-dimethylaminopyridine (DMAP), 1,4-diazabicyclo[2.2.2]octane (DABCO), proline, phenylalanine, a thiazolium salt or N,N-diisopropylethylamine (DIPEA or DIEA);
phosphorus-comprising bases, such as, for example, alkyl- and arylphosphines chosen in particular from triphenylphosphine, 2,2′-bis(diphenylphosphino)-1,1′-binaphthyl (BINAP) or triisopropylphosphine; alkyl- and arylphosphonates chosen from diphenyl phosphate, triphenyl phosphate (TPP), tri(isopropylphenyl) phosphate (TIPP), cresyl diphenyl phosphate (CDP) or tricresyl phosphate (TCP); alkyl and aryl phosphates chosen from di(n-butyl) phosphate (DBP), tris(2-ethylhexyl) phosphate or triethyl phosphate; or Verkade bases chosen from 2,8,9-triisopropyl-2,5,8,9-tetraaza-1-phosphabicyclo [3.3.3]undecane, 2,8,9-triisobutyl-2,5,8,9-tetraaza-1-phosphabicyclo [3.3.3]undecane or 2,8,9-trimethyl-2,5,8,9-tetraaza-1-phosphabicyclo [3.3.3]undecane;
carbon-comprising bases for which the protonation takes place on a carbon atom, such as, for example, a N-heterocyclic carbene, such as a carbene resulting from an imidazolium salt, for instance 1,3-bis(2,6-diisopropylphenyl)-1H-imidazol-3-ium, 1,3-bis(2,6-diisopropylphenyl)-4,5-dihydro-1H-imidazol-3-ium, 1,3-bis(2,4,6-trimethylphenyl)-1H-imidazol-3-ium, 1,3-bis (2,4,6-trimethy 1phenyl)-4,5-dihydro-1H-imidazol-3-ium, 4,5-dichloro-1,3-bis(2,6-diisopropylphenyl)-1H-imidazol-3-ium, 1,3-di(tert-butyl)-1H-imidazol-3-ium or 1,3-di(tert-butyl)-4,5-dihydro-1H-imidazol-3-ium salts, said salts being, for example, in the form of chloride salts, as represented below:

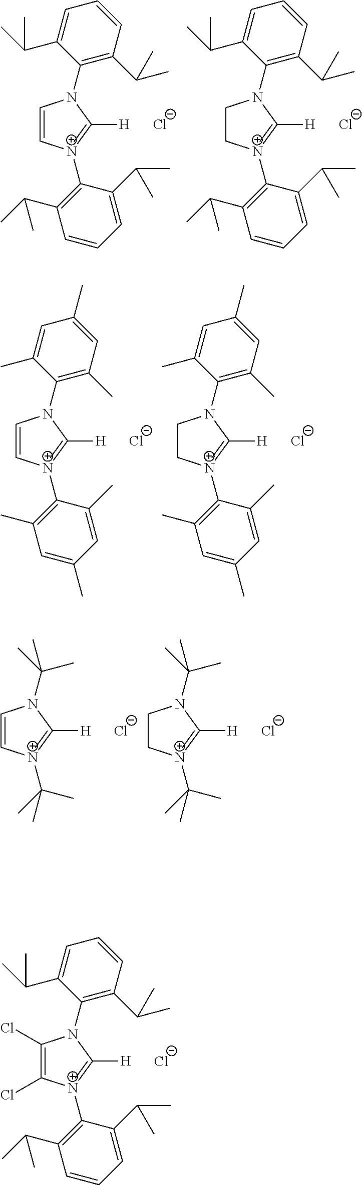

organic or inorganic fluoride salts, such as NaF, KF, CsF, TBAT (tetrabutylammonium triphenyldifluorosilicate), TASF (tris(dimethylamino)sulfonium difluorotrimethylsilicate), tetrabutylammonium fluoride or tetramethylammonium fluoride, or
oxygen-comprising bases, such as, for example, hydrogen peroxide; benzoyl peroxide; or an alkoxide, such as in particular sodium or potassium methoxide, ethoxide, propoxide, butoxide, pentoxide or hexoxide.

According to one embodiment, the catalyst is a complex of a transition metal and of an N-heterocyclic carbene.

The transition metal can in particular be chosen from nickel, iron, cobalt, zinc, copper, rhodium, ruthenium, platinum, palladium or iridium.

The N-heterocyclic carbene can in particular be a carbene resulting from an imidazolium salt, such as 1,3-bis(2,6-diisopropylphenyl)-1H-imidazol-3-ium chloride, 1,3-bis(2,6-diisopropylphenyl)-4,5-dihydro-1H-imidazol-3-ium chloride, 1,3-di(tert-butyl)-1H-imidazol-3-ium chloride or 1,3-di(tert-butyl)-4,5-dihydro-1H-imidazol-3-ium chloride.

According to a specific embodiment, the catalyst is tetrabutylammonium triphenyldifluorosilicate (TBAT) or chloro- or fluoro[1,3-bis(2,6-diisopropylphenyl)imidazol-2-ylidenelcopper (I) (respectively IPrCuCl or IPrCuF).

The catalyst can furthermore be brought together with an additive, such as an alkali metal halide, in particular cesium chloride. This additive can have the role of activating the substrate but can also make possible the regeneration of an active catalytic entity.

The catalysts can, if appropriate, be immobilized on heterogeneous supports in order to provide ready separation of said catalyst and/or its recycling. Said heterogeneous supports can be chosen from supports based on silica gel or on plastic polymers, such as, for example, polystyrene; carbon-comprising supports chosen in particular from carbon nanotubes; silicon carbide; alumina; or magnesium chloride (MgCl₂).

In the process according to the invention, the reaction can take place under a CO₂ pressure, by sparging CO₂ into the reaction medium or under a dry atmosphere containing CO₂ (dried ambient air comprising, for example, approximately 78% by volume of nitrogen, 21% by volume of oxygen, and approximately from 0.2 to 0.04% by volume of carbon dioxide). The reaction can also take place using supercritical CO₂.

Preferably, the reaction is carried out under anhydrous conditions and/or under a CO₂ pressure.

The pressure of the CO₂ can then be between 0.2×10⁵ and 50×10⁵ Pa, preferably between 10⁵ and 30×10⁵ Pa and more preferably between 10⁵ and 10×10⁵ Pa, limits included.

The temperature of the reaction can be between 25 and 150° C., preferably between 50 and 125° C. and more preferably between 70 and 100° C., limits included.

The duration of the reaction depends on the degree of conversion of the substrate of formula (Si or B). The reaction is advantageously maintained until the substrate of formula (Si or B) has been completely converted. The reaction is carried out for a period of time of 5 minutes to 72 hours, preferably of 1 to 48 hours, limits included.

The process of the invention, in particular the reaction between the different reactants, can take place in one or a mixture of at least two solvent(s) chosen from:
ethers, preferably diethyl ether or THF;
hydrocarbons, preferably benzene or toluene;
nitrogenous solvents, preferably pyridine or acetonitrile;
sulfoxides, preferably dimethyl sulfoxide;
alkyl halides, preferably chloroform or methylene chloride.

The molar ratio of the organosilane/borane of formula (Si or B) to the halide compound of formula (III) is between 0.5 to 5, preferably between 1 and 3.

The amount of catalyst is between 0.001 and 1 molar equivalent, preferably between 0.01 and 1 molar equivalent, with respect to the substrate of formula (Si or B).

Furthermore, the compounds of labeled ester type, incorporating radioisotopes and/or stable isotopes, are particularly advantageous in numerous fields, such as, for example, in life sciences (study/elucidation of enzymatic mechanisms or of biosynthetic mechanisms, in biochemistry, and the like), environmental sciences (tracing of wastes, and the like), research (study/elucidation of reaction mechanisms) or else the research and development of novel pharmaceutical and therapeutic products. Thus, to develop a synthesis for the preparation of labeled esterified compounds meeting the requirements indicated above can respond to a real need.

In addition, there exists a real need to have available a process which makes it possible to obtain, in the single stage and with an excellent selectivity, labeled esterified compounds incorporating radioisotopes and/or stable isotopes starting from labeled reactants, such as, for example, labeled CO₂ and/or labeled organosilanes/boranes and/or compounds of formula (III), under catalytic conditions.

Thus, the invention also relates to the process for the preparation of compounds of labeled ester type of formula (I′):

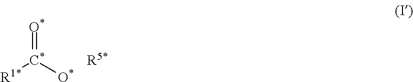

said process comprising the stages of:

a) bringing together an organosilane/borane of formula Si* or B* and C*O*₂ in the presence of a catalyst and of an electrophilic compound of formula (III′):

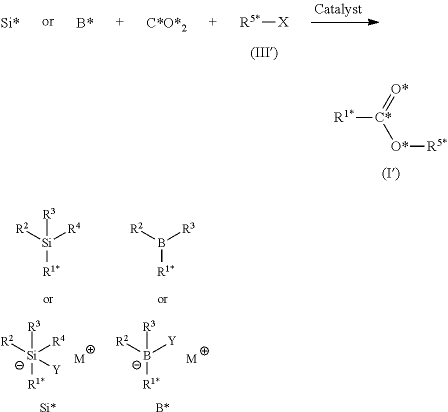

in which:

R¹* and R⁵* correspond to the R¹ and R⁵ groups as defined in the formula (I) above, and optionally comprise a H*, C*, N*, O*, F*, Si* and/or S*,

H* represents a hydrogen atom (¹H), deuterium (²H) or tritium (³H),

C* represents a carbon atom (¹²C)or a ¹¹C, ¹³C or ¹⁴C isotope,

N* represents a nitrogen atom (¹⁴N) or a ¹⁵N isotope,

O* represents an oxygen atom (¹⁶O) or a ¹⁸O isotope,

F* represents a fluorine atom (¹⁹F) or a ¹⁸F isotope,

Si* represents a silicon atom (²⁸Si) or a ²⁹Si or ³⁰Si isotope,

S* represents a sulfur atom (³²S) or a ³³S, ³⁴S or ³⁶S isotope,

it being understood that at least one of the compounds Si* or B*, C*O₂* or R⁵*—X comprises an isotope from those listed above,

R², R³, R⁴, Y, M and X are as defined in the formula (I) as above, and

b) recovering the compound of formula (I′) obtained.

Radiolabeling is the fact of combining, with a given molecule or a given compound, an isotope which will make it possible to monitor the change and/or the fixing of the molecules, for example in an organ. The radioactive tracer is the radioactive element(s) present within a molecule in order to monitor the course of this substance, for example in an organ.

This process can thus make possible access to esterified compounds labeled with ¹¹C, ¹³C, ¹⁴C and ¹⁸O.

The use of molecules for the purposes of tracing, of metabolization, of imaging, and the like, is described in detail in the literature (U. Pleiss, R. Voges, “Synthesis and Applications of Isotopically Labelled Compounds, Volume 7”, Wiley-VCH, 2001; R. Voges, J. R. Heys and T. Moenius, “Preparation of Compounds Labeled with Tritium and Carbon-14”, Wiley-VCH, Chippenham (UK), 2009).

The possibility of forming the labeled esterified compounds can be ensured by the availability of the corresponding labeled reactants, for example by:
CO₂ labeled with ¹¹C or ¹⁴C is the main source of ¹¹C and ¹⁴C is obtained by acidification of labeled barium carbonate Ba¹⁴CO₃. (R. Voges, J. R. Heys and T. Moenius, “Preparation of Compounds Labeled with Tritium and Carbon-14”, Wiley-VCH: Chippenham (UK), 2009),
alkyl or aryl halides enriched in ¹³C or labeled with ²H (deuterium or D) are commercially available.

Molecules labeled with ¹⁴C have contributed to many advances in life sciences (enzymatic mechanisms, biosynthetic mechanisms, biochemistry), environmental sciences (tracing of wastes), research (elucidation of reaction mechanisms) or else diagnosis or the research and development of novel pharmaceutical and therapeutic products. This is because molecules labeled with ¹⁴C are advantageous in metabolic studies as ¹⁴C is easily detectable and quantifiable in an in vitro and in vivo medium.

The main source of ¹⁴C is ¹⁴CO₂, which is obtained by acidification of barium carbonate Ba¹⁴CO₃. The development of processes for the synthesis of base molecules used for the preparation of medicaments is thus essential in order to produce active principles labeled with ¹⁴C, the metabolism of which can thus be determined (R. Voges, J. R. Heys and T. Moenius, “Preparation of Compounds Labeled with Tritium and Carbon-14”, Wiley-VCH: Chippenham (UK), 2009).

The major constraint limiting the synthesis of the molecules labeled with ¹⁴C is the need to have a high yield of ¹⁴C product formed, with respect to the amount of ¹⁴CO₂ used, and to be based on a restricted number of stages, in order to limit as much as possible the costs related to the use of Ba¹⁴CO₃ (U. Pleiss and R. Voges, “Synthesis and Applications of Isotopically Labelled Compounds, Volume 7”, Wiley-VCH, 2001; R. Voges, J. R. Heys and T. Moenius, “Preparation of Compounds Labeled with Tritium and Carbon-14”, Wiley-VCH: Chippenham (UK), 2009).

The process according to the invention meets these requirements as the CO₂ working pressure can be low, for example from 0.2×10⁵ to 10⁵ Pa. In addition, the degree of incorporation of CO₂ (or yield with respect to the CO₂ introduced) remains high and can, for example, exceed 95%.

According to another subject matter, the invention relates to the use of the process for the preparation of the esters of formula (I) as defined above for the recovery in value of CO₂ emissions.

Definitions

In the context of the present invention, the yield is calculated with respect to the amount of organosilane/borane of formula (Si or B) initially introduced, on the basis of the amount of ester of formula (I) isolated:

Yield (%)=n(ester)/(n(Si or B)_{initially introduced})*100, n being the amount of substance in number of moles.

In the context of the present invention, the selectivity relates to the nature of the products formed from the organosilane/borane of formula (Si or B).

“Alkyl” is understood to mean, within the meaning of the present invention, an optionally substituted, saturated, linear, branched or cyclic carbon-comprising radical comprising from 1 to 12 carbon atoms, in particular from 1 to 6 carbon atoms. Mention may be made, as saturated and linear or branched alkyl, for example, of the methyl, ethyl, propyl, butyl, pentyl, hexyl, octyl, nonyl, decyl, undecyl and dodecanyl radicals and their branched isomers. Mention may be made, as cyclic alkyl, of the cyclopropyl, cyclobutyl, cyclopentyl, cyclohexyl, bicylco[2.1.1]hexyl and bicyclo[2.2.1]heptyl radicals.

“Perfluoroalkyl” group is understood to mean a C₁-C₁₂ alkyl group in which the hydrogen atoms are replaced by fluorine atoms, of formula CₙF₂ₙ₊₁. Mention may in particular be made, as example of perfluoroalkyl group, of the trifluoromethyl (CF₃) group.

“Alkenyl” is understood to mean, within the meaning of the present invention, an optionally substituted, linear, branched or cyclic carbon-comprising radical, having at least one carbon-carbon double bond and comprising from 2 to 12 carbon atoms, in particular from 2 to 6 carbon atoms. Mention may be made, as examples of alkenyl groups, of the ethylenyl, propylenyl, butenyl, pentenyl, hexenyl and acetylenyl radicals. Mention may in particular be made, as example of cyclic alkenyl groups, of cyclopentenyl or cyclohexenyl.

“Alkynyl” is understood to mean, within the meaning of the present invention, an optionally substituted, linear, branched or cyclic carbon-comprising radical comprising at least one carbon-carbon triple bond and comprising from 2 to 12 carbon atoms, in particular from 2 to 6 carbon atoms.

The term “aryl” denotes, generally, a cyclic aromatic substituent comprising from 6 to 20 carbon atoms, in particular from 6 to 10 carbon atoms. In the context of the invention, the aryl group can be mono- or polycyclic. Mention may be made, by way of indication, of the phenyl, benzyl and naphthyl groups.

The term “arylalkyl” or “aralkyl” denotes an aryl-alkyl group in which the aryl and the alkyl are as defined above. Preferred aralkyls contain a C₁-C₄ alkyl fragment. Mention may be made, as examples of aralkyl groups, of benzyl, 2-phenethyl and naphthalenemethyl.

The term “heteroaryl” denotes, generally, a mono- or polycyclic aromatic substituent comprising from 5 to 10 members, including at least 2 carbon atoms and at least one heteroatom chosen from nitrogen, oxygen or sulfur. The heteroaryl group can be mono- or polycyclic. Mention may be made, by way of indication, of the furyl, benzofuranyl, pyrrolyl, indolyl, isoindolyl, azaindolyl, thiophenyl, benzothiophenyl, pyridyl, quinolinyl, isoquinolyl, imidazolyl, benzimidazolyl, pyrazolyl, oxazolyl, isoxazolyl, benzoxazolyl, thiazolyl, benzothiazolyl, isothiazolyl, pyridazinyl, pyrimidinyl, pyrazinyl, triazinyl, cinnolinyl, phthalazinyl or quinazolinyl groups.

The term “alkoxy” means an —O-alkyl group, the alkyl group being as defined above. Mention may in particular be made, as example of alkoxy groups, of the methoxy or ethoxy groups.

The term “heterocycle” denotes, generally, a saturated or unsaturated 5- to 10-membered mono- or polycyclic substituent containing from 1 to 4 heteroatoms chosen, independently of one another, from nitrogen, oxygen and sulfur. Mention may be made, by way of indication, of the morpholinyl, piperidinyl, piperazinyl, pyrrolidinyl, imidazolidinyl, imidazolinyl, pyrazolidinyl, tetrahydrofuranyl, tetrahydropyranyl, thianyl, oxazolidinyl, isoxazolidinyl, thiazolidinyl or isothiazolidinyl substituents.

Halogen atom is understood to mean an atom chosen from the fluorine, chlorine, bromine or iodine atoms.

Alkali metal countercation is understood to mean the lithium (Li⁺), sodium (Na⁺), potassium (K⁺), rubidium (Rb⁺) or cesium (Cs⁺) cations.

Alkaline earth metal cation is understood to mean the cations of Group II, such as magnesium (Mg²⁺) or calcium (Ca²⁺).

Catalyst is understood to mean, within the meaning of the present invention, any compound capable of modifying, in particular by increasing, the rates of the chemical reaction in which it participates and which can be regenerated at the end of the reaction or consumed during the reaction. This definition encompasses both catalysts, that it so say the compounds which exert their catalytic activity without needing to undergo any modification or conversion, and the compounds (also known as precatalysts) which are introduced into the reaction medium and which are converted into a catalyst therein.

### EXAMPLES

Abbreviations:

RT: room temperature

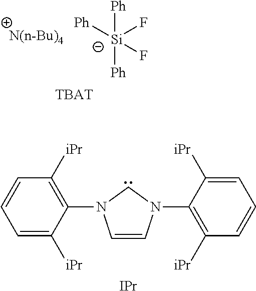

1. Materials and Methods

The catalytic reaction for the esterification of the organosilanes of the process according to the invention was carried out according to the following experimental protocol:

1. The organosilane/borane (1 equivalent), the precatalyst (from 0.001 to 1 equivalent), the additive (from 1 to 3 equivalents), the halide (from 1 to 2 equivalents) and the solvent are introduced under an inert atmosphere, in a glovebox, into a Schlenk tube, which is subsequently sealed by a J. Young valve. The concentration of organosilane/borane and of halide in the reaction mixture is approximately 0.3M (concentration calculated on the basis of the volume of solvent introduced). The order of introduction of the reactants is not important.

2. The Schlenk tube is subsequently placed under a CO₂ pressure (from 1 to 3 bar) using a vacuum line and is then heated at a temperature of between 25 and 100° C. until the organosilane/borane has completely converted (reactions of 5 minutes to 72 hours).

3. Once the reaction is complete, an Et₂O/H₂O extraction makes it possible to remove the undesirable salts. The organic phase is collected. The remaining volatile compounds in the organic phase are removed under reduced pressure and the reaction mixture is purified by chromatography on silica gel. The use of an ethyl acetate/n-pentane mixture as eluent makes it possible to obtain the analytically pure ester. Alternatively, if the boiling point of the ester of formula (I) is sufficiently low (<200° C.), the ester can be isolated from the reaction mixture by a simple distillation at ambient or reduced pressure.

2. Results

The results obtained are presented below, giving examples of conversions of various hypervalent silanes/boranes and organosilanes/boranes to give esters (determined by NMR) under stoichiometric and catalytic conditions.

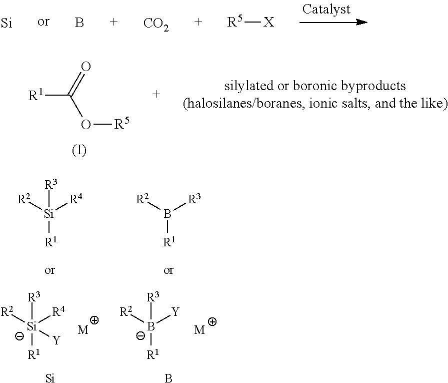

Different systems were tested for the reaction:

### 2.1. Stoichiometric Reactions Involving the Catalyst TBAT

TABLE 1

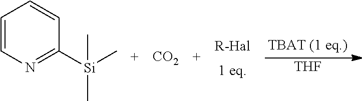

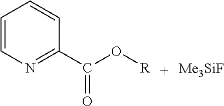

| Entry | R-Hal | Conditions | Yield (%) |
|---|---|---|---|
| 1 | n-Prl | RT, 40 h | 90 |
| 2 | i-Prl | RT, 40 h | 85 |
| 3 | n-Hexl | RT, 40 h | 93 |
| 4 | 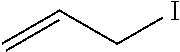 | RT, 40 h | 99 |
| 5 | 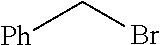 | 70° C., 23 h | 99 |
| 6 | 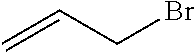 | 70° C., 3 h | 99 |
| 7 | n-HexBr | 70° C., 23 h | 93 |
| 8 | 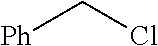 | 70° C., 23 h | 58 |
| 9 | 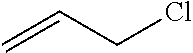 | 70° C., 3 h | 99 |
| 10 | n-HexCl | 70° C., 17 h | 99 |

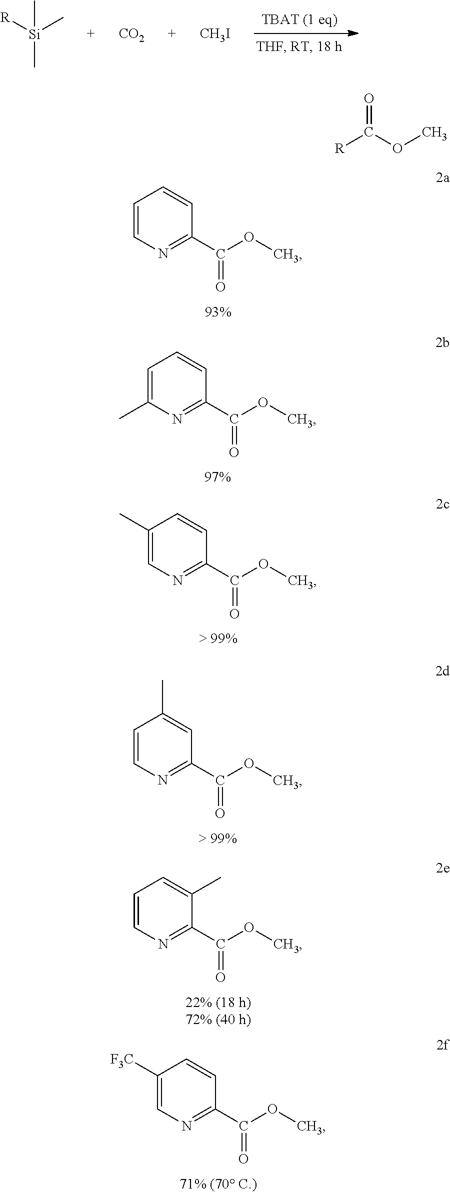

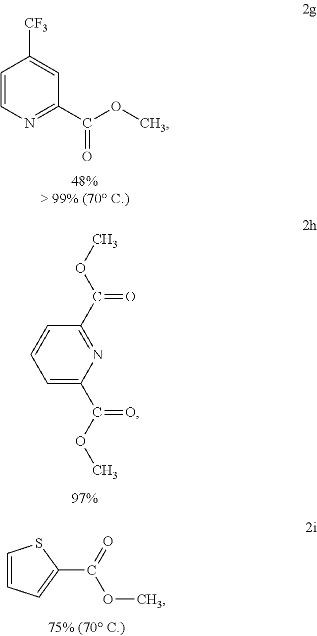

### 2.2. Copper-Based Stoichiometric Reactions

TABLE 2

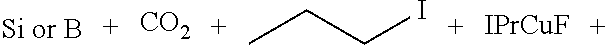

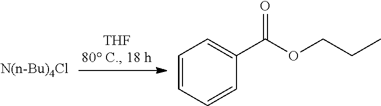

| Entry | Substrate | Yield |
|---|---|---|
| 1 | PhSi(OMe)₃ | 52 |
| 2 | PhSiMe(OEt)₂ | 46 |
| 3 | PhSi(OEt)₃ | 53 |
| 4 | p-MeOPhSi(OEt)₃ | 53 |
| 5 | p-ClPhSi(OEt)₃ | 60 |
| 6 | vinylSi(OMe)₃ | 33 |
| 7 | allylSi(OMe)₃ | 62 |

### 2.3. Copper-Based Catalytic Reactions

PySiMe₂vinyl as substrate

TABLE 3

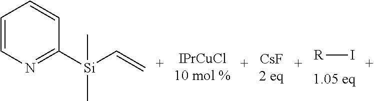

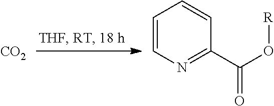

| Entry | RX | Conditions | Yield (%) |
|---|---|---|---|
| 1 | CH₃I | RT, 20 h | 86 |
| 2 | 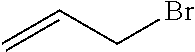 | 70° C., 20 h | 20 |
| 3 | 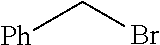 | 70° C., 20 h | 17 |
| 4 | 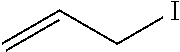 | RT, 20 h | 20 |
| 5 | n-Hexl | RT, 20 h | 34 |

TBAT as substrate

TABLE 4

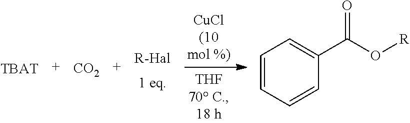

| Entry | R-Hal | Conditions | Yield (%) |
|---|---|---|---|
| 1 | n-Prl | 70° C., 18 h | 76 |
| 2 | i-Prl | 70° C., 18 h | 63 |
| 3 | n-Hexl | 70° C., 18 h | 84 |
| 4 | PhBr | 70° C., 18 h | 48 |

3. Copper-Based Catalytic Reactions with Phenyltriethoxysilane as Substrate

Variation of the Solvent

TABLE 5

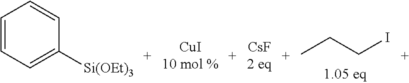

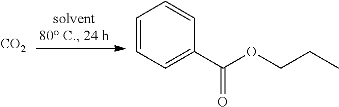

| Entry | Solvent | Yield (%) |
|---|---|---|
| 1 | THF | 22 |
| 2 | DMF | 10 |
| 3 | CH₃CN | 25 |

Variation of the Catalyst

TABLE 6

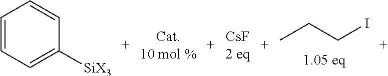

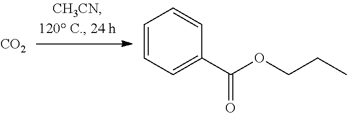

| Entry | X | Cat. | Yield (%) |
|---|---|---|---|
| 1 | OEt | CuC | 10 |
| 2 | OMe | CuCl | 10 |
| 3 | Cl | CuCl | 15 |
| 4 | F | CuCl | 82 |
| 5 | F | Cu(0) | 35 |
| 6 | F | IPrCuCl | 52 |
| 7 | F | IPr + CuF₂ | 46 |
| 8 | F | IPrCuF | 70 |
| 9 | F | SMes + CuI | 75 |
| 10 | F | itBu + CuI | 50 |
| 11 | F | SiPr + CuI | 54 |
| 12 | F | TMEDA + CuCl | 20 |
| 13 | F | Bipy + CuCl | 10 |
| 14 | F | Dppz + CuCl | 5 |

Variation of the Electrophile

TABLE 7

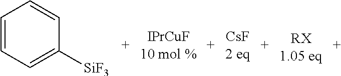

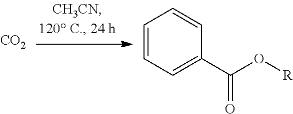

| Entry | R-Hal | Yield (%) |
|---|---|---|
| 1 | 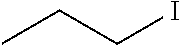 | 70 |
| 2 | 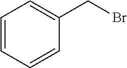 | 55 |
| 3 | 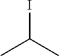 | 59 |
| 4 | 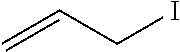 | 5 |
| 5 | 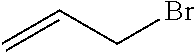 | 20 |

Variation of the Silane

TABLE 8

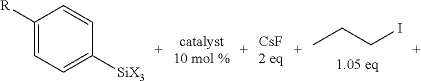

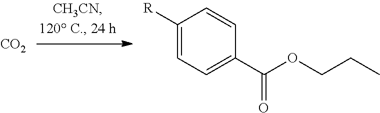

| Entry | R | X | Catalyst | Yield (%) |
|---|---|---|---|---|
| 1 | Cl | OEt | CuI | 25 |
| 2 | CF₃ | OEt | CuI | 5 |
| 3 | Me | OEt | CuI | 10 |
| 4 | Me | F | IPrCuF | 90 |

1. A process for the preparation of a carboxylic ester of formula (I): 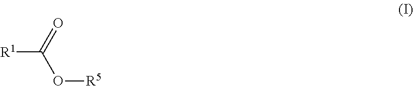
wherein:
R¹ independently represents a C₁-C₁₂ alkyl group, a C₂-C₁₂ alkenyl group, a C₂-C₁₂ alkynyl group, a C₆-C₁₀ aryl group, a (C₆-C₁₀)aryl(C₁-C₄)alkyl group, a 5- to 7-membered heteroaryl group, a 5- to 7-membered heterocycle, a silyl group —Si(R⁶)₃, a siloxy group —Si(OR⁶)₃ or an amino group —NR⁷R⁸, the alkyl, alkenyl, alkynyl, aryl, arylalkyl, heteroaryl and heterocycle groups optionally being substituted by one or more R⁹ groups,
R⁵ independently represents a C₁-C₁₂ alkyl group, a C₂-C₁₂ alkenyl group, a C₂-C₁₂ alkynyl group, a C₆-C₁₀ aryl group, a (C₆-C₁₀)aryl(C₁-C₄)alkyl group, a 5- to 7-membered heteroaryl group, a 5- to 7-membered heterocycle, a silyl group —Si(R⁶)₃, a siloxy group —Si(OR⁶)₃ or an amino group —NR⁷R⁸, the alkyl, alkenyl, alkynyl, arylalkyl, aryl, heteroaryl and heterocycle groups optionally being substituted by one or more R⁹ groups, the process comprising:
a) bringing an organosilane/borane of formula Si or B into contact with CO₂ in the presence of a catalyst and of an electrophilic compound of formula (III): 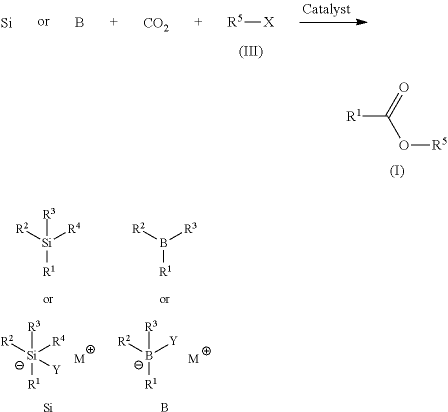
wherein:
R², R³ and R⁴ represent, independently of one another, a C₁-C₁₂ alkyl group, a C₂-C₁₂ alkenyl group, a C₂-C₁₂ alkynyl group, a C₁-C₁₂ alkoxy group, a C₆-C₁₀ aryl group, a 5- to 7-membered heteroaryl group, a 5- to 7-membered heterocycle, a silyl group —Si(R⁶)₃, a siloxy group —Si(OR⁶)₃ or an amino group —NR⁷R⁸, the alkyl, alkenyl, alkynyl, aryl, heteroaryl and heterocycle groups optionally being substituted by one or more R¹⁰ groups;
Y represents a negatively charged organic or inorganic ligand;
M represents an organic or inorganic cation;
X represents Cl, Br, I, or an —OSO₂R¹¹ group, or R⁵—X, taken in its entirety, represents an oxonium salt;
R⁶, in each case, independently represents a hydrogen atom, a halogen atom, a C₁-C₆ alkyl group, a C₁-C₆ alkoxy group or a C₆-C₁₀ aryl group;
R⁷ and R⁸ represent, independently of one another, a hydrogen atom, a C₁-C₆ alkyl group, a C₂-C₆ alkenyl group, a C₂-C₆ alkynyl group, a C₆-C₁₀ aryl group, a 5- to 7-membered heteroaryl group, a 5- to 7-membered heterocycle, a silyl group —Si(R⁶)₃ or a siloxy group —Si(OR⁶)₃;
R⁹ and R¹⁰ represent, independently of ore another a halogen atom, a C₁-C₆ alkyl group, a C₁-C₆ perfluoroalkyl group, a hydroxyl group, a C₁-C₆ alkoxy group, a nitro (—NO₂) group, a nitrile (—CN) group or a C₆-C₁₀ aryl group;
R¹¹, in each case, independently represents a C₁-C₆ alkyl or perfluoroalkyl group or a C₆-C₁₀ aryl group, the aryl group optionally being substituted by one or more C₁-C₆ alkyls,
and optionally
b) recovering the compound of formula (I) obtained.

2. The process as claimed in claim 1, wherein the catalyst is an organic catalyst, a metal salt or a metal complex.

3. The process as claimed in claim 2, wherein the catalyst is a metal salt or complex.

4. The process as claimed in claim 3, wherein the metal is selected from the group consisting of metalloids of Groups 13-16 of the Periodic Table, alkali metals, alkaline earth metals, transition metals and rare earth metals.

5. The process as claimed in claim 4, wherein the metal is silicon or copper.

6. The process as claimed in claim 1, wherein the catalyst is a metal salt or complex in which the metal is silicon or copper.

7. The process as claimed in claim 1, wherein the catalyst is a complex of a transition metal and of an N-heterocyclic carbene.

8. The process as claimed in claim 1, wherein the catalyst is tetrabutylammonium triphenyldifluorosilicate (TBAT) or chloro- or fluoro[1,3-bis(2,6-diisopropylphenyl)imidazol-2-ylidene]copper (I) (1PrCu(I)).

9. The process as claimed in claim 1, wherein in stage a) the CO₂ is under pressure.

10. The process as claimed in claim 1, wherein stage a) is carried out at a temperature of between 25° C. and 150° C.

11. The process as claimed in claim 1, wherein the molar ratio of (Si or B) to the compound of formula (Ill) is from 0.5 to 5.

12. The process as claimed in claim 1, wherein the amount of catalyst is between 0.001 and 1 molar equivalent, with respect to the substrate of formula (Si or B).

13. A process for the preparation of labeled carboxylic ester compounds of formula (I′): 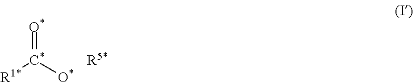
the process comprising the stages of:
a) bringing together an organosilane/borane of formula Si or B and C*O*₂ in the presence of a catalyst and of an electrophilic compound of formula (HIT 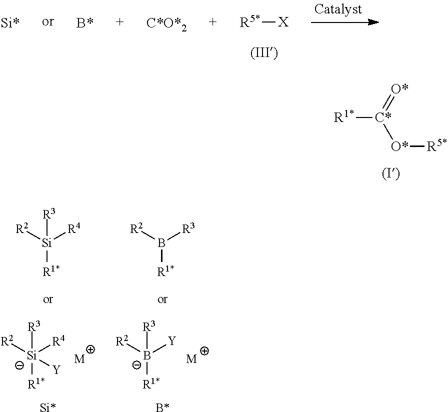
wherein:
R¹* and R⁵* respectively correspond to R¹ and R⁵ as defined in claim 1, and optionally comprise a H*, C*, N*, O*, F*, Si* and/or S*,
H* represents a hydrogen atom (¹H), deuterium (²H) or tritium (³H),
C* represents a carbon atom (¹²C) or a ¹¹C, ¹³C or ¹⁴C isotope,
N* represents a nitrogen atom (¹⁴N) or a ¹⁵N isotope,
O* represents an oxygen atom (¹⁶O) or a ¹⁸O isotope,
F represents a fluorine atom (¹⁹F) or a ¹⁸F isotope,
Si* represents a silicon atom (²⁸Si) or a ²⁹Si or ³⁰Si isotope,
S* represents a sulfur atom (³²S) or a ³³S, ³⁴S or ³⁶S isotope,
R², R³, R⁴, Y, M and X are as defined in claim 1,
where at least one of the compounds Si* or B*, C*O₂* or R⁵*—X comprises an isotope from those listed above, and optionally
b) recovering the compound of formula (I′) obtained.

14. A process as defined in claim 1 for recovery in value of CO₂ emissions.
